(19) United States
(12) Patent Application Publication
(10) Pub. No.: US 2010/0249164 A1
(43) Pub. Date: Sep. 30, 2010

(54) MODULATION OF SLEEP WITH NR2B RECEPTOR ANTAGONISTS

(76) Inventors: John J. Renger, North Wales, PA (US); Christopher J. Winrow, Doylestown, PA (US)

Correspondence Address: MERCK, P O BOX 2000, RAHWAY, NJ, 07065-0907, US

(21) Appl. No.: 12/740,030

(22) PCT Filed: Oct. 28, 2008
(86) PCT No.: PCT/US08/12214

§ 371 Date: Apr. 27, 2010

Related U.S. Application Data
(62) Division of application No. 61/001,412, filed on Oct. 31, 2007.

(51) Int. Cl. A61K 31/506; A61P 25/20; A61P 25/28; A61P 25/16; A61P 25/02; A61P 25/00

(52) U.S. Cl. 514/275

(57) ABSTRACT

An NR2B receptor antagonist is useful, alone or in combination with other agents, for promoting wakefulness, treating narcolepsy, excessive daytime sleepiness, enhancing cognition, treating sleepiness associated with Alzheimer's disease, Parkinson's disease, fibromyalgia, chronic pain, sleep disorders, autism and attention deficit hyperactivity disorder.

### BACKGROUND OF THE INVENTION

NMDA receptors are heteromeric assemblies of subunits, of which two major subunit families designated NR1 and NR2 have been cloned. It is generally believed that the various functional NMDA receptors in the mammalian central nervous system are only formed by combinations of NR1 and NR2 subunits, which respectively express glycine and glutamate recognition sites. The NR2 subunit family is in turn divided into four individual subunit types: NR2A, NR2B, NR2C, and NR2D. T. Ishii, et al., J. Biol. Chem., 268:2836-2843 (1993), and D. J. Laurie et al., Mol. Brain Res., 51:23-32 (1997) describe how the various resulting combinations produce a variety of NMDA receptors differing in physiological and pharmacological properties such as ion gating properties, magnesium sensitivity, pharmacological profile, as well as in anatomical distribution.

Although sleep is necessary for survival, its precise homeostatic contribution is unknown. Sleep is not a uniform state, but rather involves several stages characterized by changes in the individual's EEG. A non rapid eye movement (NREM) type (75 to 80% of total sleep time) ranges in depth through stages 1 to 4 (deepest level). Stage 1 sleep is drowsiness, in which the EEG displays a lower voltage, more mixed frequencies and deterioration of alpha rhythm relative to the EEG when the individual is awake. In stage 2, background activity similar to that of stage 1 is experienced, with bursts of slightly higher frequency “sleep spindles” and sporadic higher amplitude slow wave complexes. The third and fourth stages of sleep display increasing high amplitude slow wave activity. The separate sleep stage in which the individual undergoes rapid eye movement (REM) occupies the remainder of the sleep time and occurs 5 to 6 times during a normal nights sleep. REM sleep is characterized by a lower voltage, higher frequency EEG and other characteristics similar to those which occur when the individual is awake, whereas the other four sleep stages are categorized as NREM sleep.

Individuals vary widely in their requirements for sleep, which is influenced by a number of factors including their current emotional state. The natural aging process is associated with changes in a variety of circadian and diurnal rhythms. Age-related changes in the timing and structure of sleep are surprisingly common problems for older people, and are often associated with significant morbidity. With advancing age, the total amount of sleep tends to shorten. Stage 4 can decrease or disappear and sleep may become more fragmented and interrupted. Evaluation of sleep patterns in elderly people shows that the timing of sleep is also phase advanced, especially in women. This tendency to go to sleep and wake up earlier is very frustrating to older people who feel that they are out of step with the rest of the world. In addition, the quality of sleep in the elderly is diminished with a marked reduction in slow wave sleep, a reduction in the deep stages of sleep (especially stage 4), fragmentation of REM sleep and more frequent awakenings. Similarly, non-elderly people may exhibit disturbances in the normal sleep process. These changes in the structure of sleep have been correlated to more frequent napping, decreased daytime alertness and declining intellectual function and cognitive ability. Deprivation of REM sleep has been suggested to interfere with the memory consolidation involved in learning skills through repetitive activity, and slow wave sleep has been implicated as being important in consolidation of events into long term memory. Likewise, decreases in the length of REM stages of sleep may be associated with a decrease in cognitive function and learning, especially diminished retention of memory.

Sleep disorders generally involve disturbances of sleep that affect a subject's ability to fall and/or stay asleep, and involve sleeping too little, too much or resulting in abnormal behavior associated with sleep. Various compounds are known to improve wakefulness in humans. For example, U.S. Patent Application Publication No. 20040143021 describes the use of modafinil to improve wakefulness following the administration of general anesthesia. U.S. Patent Application Publication No. 20010034373 similarly describes the administration of modafinil to improve cognitive function.

Currently available drugs used to modulate wakefulness and sleep, such as drugs that induce sleep, prolong wakefulness, or enhance alertness, suffer from a number of shortcomings. For example, available sleep-inducing drugs often do not achieve the fully restorative effects of normal sleep. Often such drugs cause undesirable effects upon waking, such as anxiety or continued sedation. Furthermore, many of the currently available drugs that modulate sleep and wakefulness are addictive or have adverse effects on learning and memory. Thus, there exists a need to identify new therapeutic agents that can be used to promote wakefulness and sleep.

### SUMMARY OF THE INVENTION

The present invention is directed to the use of a compound which has the ability to antagonize NR2B receptors for promoting wakefulness, treating narcolepsy, excessive daytime sleepiness, enhancing cognition, treating sleepiness associated with Alzheimer's disease, Parkinson's disease, fibromyalgia, chronic pain, sleep disorders, autism and ADHD, diseases wherein excessive sleepiness is a contributing factor or a complicating condition associated with another disease or following anesthesia, in a human. The present invention provides a method for promoting wakefulness, treating narcolepsy, excessive daytime sleepiness, enhancing cognition, treating sleepiness associated with Alzheimer's disease, Parkinson's disease, fibromyalgia, chronic pain, sleep disorders, autism and ADHD, diseases wherein excessive sleepiness is a contributing factor or a complicating condition associated with another disease or following anesthesia in a patient in need thereof that comprises administering to the patient a therapeutically effective amount of an NR2B receptor antagonist or a pharmaceutically acceptable salt thereof, alone or in combination with other agents.

### DESCRIPTION OF THE INVENTION

The present invention provides a method for the use of an NR2B receptor antagonist to promote wakefulness, treating narcolepsy, excessive daytime sleepiness, enhancing cognition, treating sleepiness associated with Alzheimer's disease, Parkinson's disease, fibromyalgia, chronic pain, sleep disorders, autism and ADHD, diseases wherein excessive sleepiness is a contributing factor or a complicating condition associated with another disease or following anesthesia, in a human.

The present invention further provides a method for enhancing the state of wakefulness, alertness, and/or central nervous system stimulation, promoting wakefulness, treating narcolepsy, excessive daytime sleepiness, excessive sleepiness associated with narcolepsy, obstructive sleep apnea/hypopnea syndrome, jet lag or wakefulness disturbances as a consequence of jet lag, or other disorders (including diseases of the nervous system), e.g., hypersomnia, REM behavior disorder, frontal nocturnal dystonia, restless legs syndrome, insomnia, parasomnia, nocturnal epileptic seizure, nocturnal movement disorder, sleep-related diagnostic dilemma, sleep apnea associated with neurological disorders, shift worker sleep disorder (SWSD), Kleine-Levin syndrome, sleep/wake disorders in blind subjects, and Parkinsonism, sleep disturbances induced by neurological injuries, abnormalities, lesions, or surgery, treating sleepiness associated with Alzheimer's disease, Parkinson's disease, fibromyalgia, chronic pain, sleep disorders, autism and ADHD, treating diseases wherein excessive sleepiness is a contributing factor or a complicating condition associated with another disease or following anesthesia, and enhancing cognition in a patient in need thereof that comprises administering to the patient a therapeutically effective amount of an NR2B receptor antagonist or a pharmaceutically acceptable salt thereof, alone or in combination with other sleep or wake promoting agents.

The present invention further provides a method for treating, preventing or managing a sleep disorder comprising administering to a patient in need thereof a therapeutically effective amount of an NR2B receptor antagonist or a pharmaceutically acceptable salt thereof. The present invention further provides a method for the treatment of a patient suffering from a sleep disorder, such as insomnia, sleep apnea, periodic limb movements, restless leg syndrome, narcolepsy, and problem sleepiness, or for increasing cognitive function in sleep deprived patient comprising administering to a patient in need thereof a therapeutically effective amount of an NR2B receptor antagonist or a pharmaceutically acceptable salt thereof. The present invention further provides a method for the treatment of a patient suffering from excessive sleepiness associated with narcolepsy, obstructive sleep apnea/hypopnea syndrome, shift work sleep disorders desynchronization disorders, ovulation disorders, seasonal melancholia, jet lag (time zone syndrome) or wakefulness disturbances as a consequence of jet lag, or diseases of the nervous system sleep disorder comprising administering to a patient in need thereof a therapeutically effective amount of an NR2B receptor antagonist or a pharmaceutically acceptable salt thereof.

The present invention further provides a method for modulating circadian rhythmicity dysfunctions due to shift work, aging, blindness, jet-lag, exposure to sub-arctic days and nights, or other environmental circumstances in a patient in need thereof comprising comprising administering to the patient a therapeutically effective amount of an NR2B receptor antagonist or a pharmaceutically acceptable salt thereof. The present invention further provides a method for the treatment of a patient suffering from cognitive disorders, brain trauma, depression, motion sickness and vertigo, disorders of sleep and wakefulness such as narcolepsy, shift-work syndrome, drowsiness as a side effect from a medication, maintenance of vigilance to aid in completion of tasks and the like, cataplexy, hypersomnia, somnolence syndrome, jet lag, sleep apnea and the like, attention deficit hyperactivity disorder (ADHD), schizophrenia, dementia, Alzheimer's disease, circadian rhythm sleep disorders, shift work sleep disorder, and periodic limb movement disorder comprising administering to a patient in need thereof a therapeutically effective amount of an NR2B receptor antagonist or a pharmaceutically acceptable salt thereof.

The present invention further provides a method for: increasing active wake during a sleep period; increasing active wake during a day active period; decreasing light sleep during a sleep period; decreasing delta sleep during a sleep period; decreasing delta sleep during a day active period; increasing REM sleep during a sleep period; and/or decreasing REM sleep during a day active period, comprising administering to a patient in need thereof a therapeutically effective amount of an NR2B receptor antagonist or a pharmaceutically acceptable salt thereof.

Increased wakefulness may be desired in an individual having sleepiness, a tendency to fall asleep, or having a sense of excessively deep sleep. A need for wakefulness can arise in an individual having or absent of a sleep-related disorder. For example, an individual may desire wakefulness to enhance performance in mental or physical activities, such as long distance driving, shift work and study. A need for wakefulness can also be caused by a disorder of excessive daytime sleepiness. Sleep apnea, narcoplepsy, idiopathic hypersomnia and psychogenic hypersomnia are examples of common disorders which lead to daytime sleepiness. Other causes of daytime sleepiness include, for example, sleep apnea, obesity, sleep deprivation, and adverse drug reactions.

Idiopathic hypersomnia is a disorder of excessive diurnal and nocturnal sleep characterized by virtually constant sleepiness, lengthy but nonrefreshing naps, prolonged night sleep, major difficulty with morning awakening, and sometimes sleep drunkeness. Idiopathic hypersomnia appears to have familial incidence and is associated with the presence of the HLA antigen HLA-DRS. Therefore, those skilled in the art will understand that it will be possible to determine susceptibility to hypersomnia by genetic or biochemical profile.

Narcolepsy is characterized by excessive daytime sleepiness, often with involuntary symptoms of reduced wakefulness including, for example, cataplexy, which is muscle weakness or paralysis in response to sudden emotion, sleep paralysis, which is the inability to move or call out when first awake, and hallucinations. Pharmacologically, treatment of narcolepsy involves separate treatments for sleep attacks and cataplexy. Epidemiological studies have identified several predictive factors for the development of narcolepsy, including a history of cataplexy. A genetic predisposition to narcolepsy can be indicated by the presence of HLA allele DQB 10602. Therefore, those skilled in the art understand that it will be possible to determine susceptibility to narcolepsy by genetic or biochemical profile.

As used herein, the term “promoting wakefulness” refers to a decrease in sleepiness, tendency to fall asleep, or other symptoms of undesired or reduced alertness or consciousness compared with sleepiness, tendency to fall asleep, or other symptoms of undesired or reduced alertness or consciousness expected or observed without treatment. Promoting wakefulness refers to a decrease in any stage of sleep, including light sleep, deeper sleep characterized by the presence of high amplitude, low wave brain activity termed “slow wave sleep”, and rapid eye movement (REM) sleep. A compound that promotes wakefulness can, for example, cause a patient to wake from sleep, prolong periods of wakefulness, prolong normal latency to sleep, restore normal sleep patterns following sleep deprivation, or enhance beneficial wake-like characteristics, such as alertness, responsiveness to stimuli, and energy.

As used herein, the term “sleep disorder” refers to a disorder that manifests symptoms which include abnormal sleep cycles, e.g., difficulty in falling and staying asleep, difficulty in staying awake, sleep fragmentation, irregularities in sleep/wake cycle, and excessive day time sleepiness. Specific examples of sleep disorders include, but are not limited to, those listed in ICSD-R (2001). Sleep disorders that can be treated, prevented or managed using the compounds of this invention include, but are not limited to, those listed in ICSD Manual (2001). Specific examples of sleep disorders include, but are not limited to, dyssomnias and parasomnias. Examples of dyssomnias include, but are not limited to: circadian rhythm sleep disorders such as advanced sleep-phase syndrome, delayed sleep phase syndrome, irregular sleep/wake pattern, non-24-hour sleep/wake disorder, shift-work sleep disorder, sleep rhythm reversals, time-zone change syndrome and other circadian rhythm sleep disorders known in the art; extrinsic sleep disorders such as adjustment sleep disorder, alcohol-dependent sleep disorder, altitude insomnia, environmental sleep disorder, inadequate sleep hygiene, insufficient sleep syndrome, limit-setting sleep disorder, sleep-onset association disorder, stimulant dependent sleep disorder, toxin-induced sleep disorder and other extrinsic sleep disorders known in the art; and intrinsic sleep disorders such as central alveolar hypoventilation, idiopathic insomnia, narcolepsy, obstructive sleep apnea syndrome, periodic limb movement disorder, posttraumatic hypersomnia, psychophysiological insomnia, recurrent hypersomnia, sleep state misperception and other intrinsic sleep disorders known in the art. In a specific embodiment, the sleep disorder is not restless leg syndrome.

By the term “NR2B receptor antagonist” is meant any exogenously administered compound or agent that directly or indirectly antagonizes the activity of the NR2B subunit of the NMDA receptor in an animal, in particular, a human.

The NR2B receptor antagonist may be peptidal or non-peptidal in nature, however, the use of a non-peptidal NR2B receptor antagonist is preferred. In addition, for convenience the use of an orally active NR2B receptor antagonist is preferred. In an alternate embodiment, the NR2B receptor antagonist inhibits NR2B receptors during the day or following sleep, especially in the first half of the day or of the wake cycle, and even more especially in the first few hours following awakening.

In an embodiment of the present invention the NR2B receptor antagonist is a selective antagonist of the NR2B receptor.

In an embodiment of the present invention the NR2B receptor antagonist has a pharmacological half life (T½ life) in humans of ultra short duration. In another embodiment of the present invention the NR2B receptor antagonist has a pharmacological half life (T % life) in humans of short duration. In another embodiment of the present invention the NR2B receptor antagonist has a pharmacological half life (T½ life) in humans of intermediate duration. In another embodiment of the present invention the NR2B receptor antagonist has a pharmacological half life (T½ life) in humans of long duration. In another embodiment of the present invention the NR2B receptor antagonist has a pharmacological half life (T½ life) in humans of at least about 2 hours duration, but less than about 6 hours duration. In another embodiment of the present invention the NR2B receptor antagonist has a pharmacological half life (T½ life) in humans of at least about 3 hours duration, but less than about 5 hours duration.

Representative NR2B receptor antagonists are disclosed in e.g., U.S. Pat. Nos. 7,259,157, 7,217,716, 7,053,089, 7,019,016, 6,958,351, 6,919,355, 6,896,872, 6,495,561, 6,489,477, 6,476,041, 6440,976, 6,432,976, 6,380,205, 6,376,530, 6,369,076, 6,362,196, 6,316,474, 6,291,499 and PCT Patent Publications WO2007/063839, WO2007/063286, WO2007/099828, WO2006/010967, WO2006/137465, WO2006/017409, WO2007/006157, WO2006/113471, WO2005/080317, WO2006/017409, WO2006/010967, WO2006/010966, WO2006/010965, WO2006/010964, WO2004/089366, WO2005/102390, WO2005/097782, WO2005/080317, WO2005/035523, WO2005/035522, WO2005/030720, WO2005/019221, WO2005/019222, WO2004/108705, WO2004/048364, WO2003/084931, WO2002/000629, WO2002/100352, WO2002/080928, WO2002/068409, WO2002/000629, WO2001/030330, WO2001/032634, WO2001/032615, WO2001/032179, WO2001/032177, WO2001/032174, WO2001/032171, WO2001/030330, WO2000/067755.

For use in medicine, the salts of the compounds employed in this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free base with a suitable organic or inorganic acid. Representative salts include the following: Acetate, Benzenesulfonate, Benzoate, Bicarbonate, Bisulfate, Bitartrate, Borate, Bromide, Calcium, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Subacetate, Succinate, Sulfate, Sulfonate, Tannate, Tartrate, Teoclate, Tosylate, Triethiodide and Valerate. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds employed in the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers.

The identification of a compound as an NR2B receptor antagonist may be readily determined without undue experimentation by methodology well known in the art, including the “NR2B Calcium Flux Assay” and/or the “NR2B Binding Assay.” The ability of a compound to inhibit NR1a/NR2B NMDA receptor, as measured by NR1a/NR2B receptor-mediated Ca²⁺ influx, is determined by the following calcium flux assay procedure: NR1a/NR2B receptor transfected L(tk-) cells were plated in 96-well format at 3×10⁴ cells per well and grown for one to two days in normal growth medium (Dulbeccos MEM with Na pyruvate, 4500 mg glucose, pen/strep, glutamine, 10% FCS and 0.5 mg/mL geneticin). NR1a/NR2B-expression in these cells was induced by the addition of 4-20 nM dexamethasone in the presence of 500 μM ketamine for 16-24 hours. Solutions of NR2B antagonists were prepared in DMSO and serially diluted with DMSO to yield 10 solutions differing by 3-fold in concentration. A 96-well drug plate was prepared by diluting the DMSO solution 250-fold into assay buffer (Hanks Balanced Salt Solution (HBSS) Mg²⁺ free (Gibco #14175-079) containing 20 mM HEPES, 2 mM CaCl₂, 0.1% BSA and 250 μM Probenecid (Sigma #P-8761)). After induction, the cells were washed twice (Labsystem cell washer, 3 fold dilutions leaving 100 μL) with assay buffer and loaded with 4 μM of the calcium fluorescence indicator fluo-3 AM (Molecular Probes #P-1241) in assay buffer containing Pluronic F-127 (Molecular Probes #P-3000) and 10 μM ketamine at 37° C. for one hour. The cells were then washed eight times with assay buffer leaving 100 μL of buffer in each well. Fluorescence intensity was immediately measured in a FLIPR (Fluorometric Imaging Plate Reader, Molecular Devices) using an excitation of 488 nm and emission at 530 nm. Five seconds after starting the recording of fluorescence intensity, 50 μL of agonist solution (40 μM glutamate/glycine, the final concentration 10 μM) was added and after one minute, when fluorescence signal was stable, 50 μl of NR2B antagonists and control solutions from the drug plate were added and the fluorescence intensity recorded for another 30 minutes. The IC₅₀ values were determined by a non-linear least squares fitting of the endpoint fluorescence values to Equation #1:

$$Endpoint Florescence = \frac{( Ymax - Ymin )}{1 + {( [ Drug ] / {IC}_{50} )}^{nH}} Ymin$$

where, Ymin is average endpoint fluorescence of the control wells containing 1 μM of AMD-2 and Ymax is the average endpoint fluorescence of wells containing 0.1% DMSO in assay buffer.

The radioligand NR2B binding assay was performed at room temperature in 96-well microtiter plates with a final assay volume of 1.0 mL in 20 mM Hepes buffer (pH 7.4) containing 150 mM NaCl. Solutions of NR2B antagonists were prepared in DMSO and serially diluted with DMSO to yield 20 μL of each of 10 solutions differing by 3-fold in concentration. Non-specific binding (NSB) was assessed using AMD-1 (10 μM final concentration), and total binding (TB) was measured by addition of DMSO (2% final concentration). Membranes expressing NR1a/NR2B receptors (40 μM final concentration) and tritiated AMD-2 (1 nM final concentration) were added to all wells of the microtiter plate. AMD-1 can be synthesized according to the general procedure described by C. F. Claiborne et al (Bioorganic & Med. Chem. Letters 13, 697-700 (2003) and tritiated AMD-2 can be synthesized according to the general procedure disclosed in e.g., U.S. Pat. No. 7,259,157. After 3 hours of incubation at room temperature, samples are filtered through Packard GF/B filters (presoaked in 0.05% PEI, polyethyleninine Sigma P-3143) and washed 10 times with 1 mL of cold 20 mM Hepes buffer per wash. After vacuum drying of the filter plates, 40 μL of Packard Microscint-20 was added and bound radioactivity determined in a Packard TopCount. The apparent dissociation constant (K_{I}), the maximum percentage inhibition (% Iₘₐₓ), the minimum percentage inhibition (% Iₘᵢₙ) and the hill slope (nH) were determined by a non-linear least squares fitting the bound radioactivity (CPM bound) to Equation #2:

$$CPM Bound = \frac{( SB ) ( \% {I}_{max} - \% {I}_{min} ) / 100}{( 1 + {( [ Drug ] / ( {K}_{1} ( 1 + [ AMD - 2 ] / {K}_{D} ) ) )}^{nH} )} + NSB + ( SB ) ( 100 - \% {I}_{max} ) / 100$$

where, K_{D} is the apparent dissociation constant for the radioligand for the receptor as determined by a hot saturation experiment and SB is the specifically bound radioactivity determined from the difference of TB and NSB control wells.

The intrinsic NR2B receptor antagonist activity of a compounds which may be used in the present invention may be determined by these assays.

The term “therapeutically effective amount” shall mean that amount of a drug or pharmaceutical agent that will elicit the biological or medical response of a tissue, system, animal or human that is being sought by a researcher or clinician.

Accordingly, the present invention includes within its scope the use of an NR2B receptor antagonist, alone or in combination with other agents, for the prevention or treatment of sleep disorders and sleep disturbances in a warm-blooded animal. For the purposes of this disclosure, a warm-blooded animal is a member of the animal kingdom which includes but is not limited to mammals and birds. The preferred mammal for purposes of this invention is human.

The subject treated in the present methods is generally a mammal, preferably a human being, male or female, in whom antagonism of NR2B receptor activity is desired. In the present invention, it is preferred that the subject mammal is a human. Although the present invention is applicable both old and young people, it would find greater application in elderly people. Further, although the invention may be employed to enhance the sleep of healthy people, it may be especially beneficial for enhancing the sleep quality of people suffering from sleep disorders or sleep disturbances. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The present invention includes within its scope a pharmaceutical composition for enhancing and improving the quality of sleep comprising, as an active ingredient, at least one NR2B receptor antagonists in association with a pharmaceutical carrier or diluent. Optionally, the active ingredient of the pharmaceutical compositions can comprise another agent in addition to at least one NR2B receptor antagonist to minimize the side effects or with other pharmaceutically active materials wherein the combination enhances efficacy and minimizes side effects.

The present invention is further directed to a method for the manufacture of a medicament for enhancing sleep, augmenting sleep, improving the quality of sleep and for the treatment of sleep disorders and sleep disturbances in humans by employing a compound that is an NR2B receptor antagonist with a pharmaceutical carrier or diluent.

It will be known to those skilled in the art that there are numerous compounds now being used in an effort to enhance and improve the quality of sleep. Combinations of these therapeutic agents some of which have also been mentioned herein with an NR2B receptor antagonist will bring additional, complementary, and often synergistic properties to enhance the desirable properties of these various therapeutic agents. In these combinations, the NR2B receptor antagonist and the therapeutic agents may be independently present in dose ranges from one one-hundredth to one times the dose levels which are effective when these compounds are used singly.

In accordance with the present invention, an NR2B receptor antagonist may be co-administered in effective dosages with effective dosages of sleep-inducing agents in order to modulate the amount and/or timing of wake and sleep, for example in the case of circadian rhythmicity dysfunctions due to shift work, aging, blindness, jet-lag, exposure to sub-arctic days and nights, or other environmental circumstances. In this context, the sleep-inducing agent would be administered to promote sleep at an appropriate time and the wakefulness promoting agent would be administered to promote wakefulness at the appropriate time, thereby modifying the patient's sleep-wake cycle. For such uses, the sleep-inducing agent and the wakefulness-promoting agent can be packaged together, e.g., in “day-night” packaging so that it is convenient for the patient to know which drug to use at what time of the day. Examples of other sleep-inducing agents include among others melatonin agonists, eszopiclone, zolpidem, zopiclone, brotizolam and triazolam. In accordance with the present invention, an NR2B receptor antagonist may also be co-administered in effective dosages with effective dosages of other wakefulness-promoting agents to enhance the wakefulness promoting or other effects. For example, the wakefulness promoting agents of the invention can be co-administered with caffeine, modafinil, armodafinil, stimulants, amphetamines, sodium oxybate, CX516, CX517, CEP-16795, PD6735, JNJ-5207852, JNJ-7737782, JNJ-17216498, VSF-173, PH15, delayed onset sleep medications, GABA modulators, histamine receptor antagonists, histamine receptor inverse agonists, orexin receptor antagonist, T-type calcium channel antagonists, pain medications and L-Dopa, to enhance or complement the effects of both agents.

Similarly, the NR2B receptor antagonist may be administered in combination with sedatives, hypnotics, anxiolytics, antipsychotics, antianxiety agents, minor tranquilizers, melatonin agonists and antagonists, melatonergic agents, benzodiazepines, barbiturates, 5HT-2 antagonists, and the like, or the NR2B receptor antagonist may be administered in conjunction with the use of physical methods such as with light therapy or electrical stimulation. For example, to enhance and improve the quality of sleep an NR2B receptor antagonist may be given in combination with such compounds as: adinazolam, allobarbital, alonimid, alprazolam, amitriptyline, amobarbital, amoxapine, bentazepam, benzoctamine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, as well as admixtures and combinations thereof. To illustrate these combinations, an NR2B receptor antagonist effective clinically effective clinically at a given daily dose range may be effectively combined, at levels which are equal or less than the daily dose range, with such compounds at the indicated per day dose range. Typically, the individual daily dosages for these combinations may range from about one-fifth of the minimally recommended clinical dosages to the maximum recommended levels for the entities when they are given singly. It will be readily apparent to one skilled in the art that the NR2B receptor antagonist may be employed with other agents to control sleep disorders and sleep disturbances in depressed patients and/or provide benefit in the prevention or treatment of sleep disorders and sleep disturbances.

The NR2B receptor antagonist may be used alone or in combination with other NR2B receptor antagonists or with other agents which are known to be beneficial in the enhancement of sleep efficiency. The NR2B receptor antagonist and the other agent may be co-administered, either in concomitant therapy or in a fixed combination. For example, the NR2B receptor antagonist may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the NR2B receptor antagonist may be administered in conjunction with the use of physical methods such as with light therapy or electrical stimulation.

Naturally, these dose ranges may be adjusted on a unit basis as necessary to permit divided daily dosage and, as noted above, the dose will vary depending on the nature and severity of the disease, weight of patient, special diets and other factors.

These combinations may be formulated into pharmaceutical compositions as known in the art and as discussed below. An NR2B receptor antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. Illustrative of the adjuvants which may be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. In the case of capsules, tablets and pills, the dosage forms may also comprise buffering agents. When the unit dosage form is a capsule, it may contain, in addition to materials of the above type, a liquid carrier such as fatty oil. Various other materials may be present as coatings or to otherwise modify the physical form of the dosage unit. Tablets and pills can additionally be prepared with enteric coatings and tablets may be coated with shellac, sugar or both.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring, and perfuming agents. A syrup or elixir may contain the active compound, sucrose as a sweetening agent, methyl and propyl parabens as preservatives, a dye and a flavoring such as cherry or orange flavor.

Preparations according to this invention for parenteral administration include sterile aqueous or non-aqueous solutions, suspensions, or emulsions. Sterile compositions for injection may be formulated according to conventional pharmaceutical practice. Compositions for sublingual administration are also prepared with standard excipients well known in the art.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. For best results, the sleep-inducing agent should be administered within about an hour of bedtime and the wakefulness-inducing agent administered within about an hour of wakefulness or about 8 to about 10 hours post-sleep inducing agent administration. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans, to obtain effective antagonism of NR2B receptor. The dosage range will generally be about 0.5 mg to 1.0 g. per patient per day which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Pharmaceutical compositions of the present invention may be provided in a solid dosage formulation preferably comprising about 0.5 mg to 500 mg active ingredient, more preferably comprising about 1 mg to 250 mg active ingredient. The pharmaceutical composition is preferably provided in a solid dosage formulation comprising about 1 mg, 5 mg, 10 mg, 25 mg, 50 mg, 100 mg, 200 mg or 250 mg active ingredient. The following examples are provided for the purpose of further illustration only and are not intended to be limitations on the disclosed invention.

### Example 1

### Preclinical Study of an NR2B Receptor Antagonist

Surgical Procedures: Eight adult male Sprague Dawley rats (450-600 g; Taconic Farms, Germantown, N.Y.) were subcutaneously implanted with telemetric physiologic monitors (Model F50-EEE; Data Sciences International, Arden Hills, Minn.) that were used to simultaneously record both the electrocorticogram (ECoG) and electromyogram (EMG) activities of the rat. (Renger et al., 2004). Briefly, animals were anesthetized with isoflurane and electrodes for recording ECoG signals and EMG signals were placed. For placement of ECoG wire leads, holes slightly larger than the coil diameter of the transmitter lead wire were drilled in the skull 2 mm on either side of midline suture and 2 mm anterior to the lambda suture. The exposed lead wires were placed between the skull and underlying dura. A single ground electrode was placed under the skull, directly over the orbital sinus. Wires were secured to the skull with dental acrylic. EMG leads were then placed in the body of a dorsal neck muscle and secured with sutures. The signal transmitter body was placed subcutaneously over the dorsal thorax. The animals were given a single dose of antibiotic (gentomycin, 5.8 mg/kg) and an analgesic (buprenorphine, 0.1 ml) within 3 hours following surgery. The animals were allowed to recover from surgery for at least two weeks prior to recording. Throughout these experiments, animals were housed individually in plastic cages (19″×10½″×8″; Lab Products, Seaford, Del.) and were provided water and food ad libitum. Lights were on a 12 hour light: 12 hour dark cycle with lights off at 4:00 a.m. and on at 4:00 p.m.

Compound administration: Compounds were administered approximately 60 minutes prior to lights on. Recordings were started just prior to compound administration and were collected for at 16 hours. The NR2B receptor antagonist was acutely dissolved in 0.5% methylcellulose and administered by oral gavage in 1 ml total volume at a final dose of 25 mg/kg. The experiments were based on a standard cross-over design with 4 animals receiving compound for one week and the complementary group receiving vehicle (0.5% methylcellulose), followed by a week of reversed administration. All animals were exposed to two days administration of orally gavaged vehicle prior to initiation of experimental drug administration. This allowed for habituation to this treatment to occur.

Automated sleep staging and data analysis: Signals were collected simultaneously from all animals with Dataquest ART software system (Data Sciences International, Arden Hills, Minn.) at 500 Hz and stored on a PC for off-line analysis. For baseline sleep measurements continuous recordings were collected for approximately four days to get average sleep behaviors for each animal over contiguous days prior to drug and vehicle administration. During the drug administration studies, recordings were collected each day prior to, during, and following drug administration. Recordings were begun prior to compound administration so that the exact time of administration was recorded within the raw data file as artifactual noise which was caused by removing the implanted transmitter from the receptive field of the receiver during administration. This information allowed a direct measure of drug/vehicle administration time during offline analysis and was not included in the data analysis. Following the completion of data collection, all data were scored with automated sleep stage analysis software, Somnologica (Medcare Co.; Buffalo, N.Y.). Assignment of sleep stages was made. Sleep stages were assigned based upon a combination of level of movement within the field of the radio frequency receiver over which individually housed rats were caged, EMG activity, and ECoG frequencies over 10 second epochs. Active wake was assigned to the epoch when movement of the animal was detected over the receiver, or when there was an active EMG signal over the epoch and the ECoG frequencies consisted of low-voltage high frequency activity. An epoch was scored as light sleep when there was no movement activity, the EMG was moderately active and the ECoG consisted of either theta or theta activity mixed with less than 50% of the epoch showing delta activity. Delta sleep was scored when there was no gross movement, reduced EMG activity, and the ECoG consisted of more than 50% delta wave activity (i.e. 0.5 to 4 Hz). Rapid eye movement (REM) sleep was scored when there was no movement or EMG activity, and the ECoG consisted of primarily theta activity. Results of staging were grouped into 30 minute periods following drug administration and the number of entries into each stage and the duration of minutes spent in each stage were calculated. The results for all eight animals were averaged by treatment, or vehicle, over seven administration nights and the results were statistically compared based upon a two-tailed Student's t-test for each thirty minute period.

The NR2B receptor antagonist

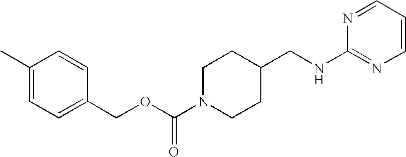

was active in this preclinical rat sleep model. This compound was prepared as described in Example 17 of U.S. Pat. No. 7,053,089. In this study, the NR2B receptor antagonist: increased active wake at the start of the sleep period and 30 minutes during the following day active period; decreased light sleep for the first half of the sleep period; decreased delta sleep at the start of the sleep period and for 1 hour during the following day active period; and increase REM sleep for the second half of the sleep period and decreased REM sleep for 30 minutes during the following day active period. These studies demonstrate that NR2B receptor antagonists have activity in promoting wakefulness that is comparable to the marketed compound modafinil.

### Example 2

### Clinical Study of an NR2B Receptor Antagonist in Healthy Young Adults

In this study, 9 healthy young men (ages 18 to 30 years) who did not suffer sleep complaints are randomly assigned to a sequence of 3 treatment periods. In each period the subjects received a single oral dose of either placebo, 5 mg of NR2B receptor antagonist or 25 mg NR2B receptor antagonist once daily for 7 days. Sleep is recorded for 2 nights: a habituation night and a blood sampling night (Days 6 and 7 of study drug administration). This study may be used to assess the efficacy of an NR2B receptor antagonist in promoting wakefulness and treating sleep disorders in humans.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for promoting wakefulness; treating narcolepsy; treating excessive daytime sleepiness; enhancing cognition; or treating sleepiness associated with Alzheimer's disease, Parkinson's disease, fibromyalgia, chronic pain, sleep disorders, autism or attention deficit hyperactivity disorder, in a patient in need thereof that comprises administering to the patient a therapeutically effective amount of an NR2B receptor antagonist or a pharmaceutically acceptable salt thereof.

2. The method of claim 1 which is a method for promoting wakefulness in a patient in need thereof that comprises administering to the patient a therapeutically effective amount of an NR2B receptor antagonist or a pharmaceutically acceptable salt thereof.

3. The method of claim 1 which is a method for treating narcolepsy in a patient in need thereof that comprises administering to the patient a therapeutically effective amount of an NR2B receptor antagonist or a pharmaceutically acceptable salt thereof.

4. The method of claim 1 which is a method for treating excessive daytime sleepiness in a patient in need thereof that comprises administering to the patient a therapeutically effective amount of an NR2B receptor antagonist or a pharmaceutically acceptable salt thereof.

5. The method of claim 1 which is a method for enhancing cognition in a patient in need thereof that comprises administering to the patient a therapeutically effective amount of an NR2B receptor antagonist or a pharmaceutically acceptable salt thereof.

6. The method of claim 1 which is a method for treating sleepiness associated with Alzheimer's disease, Parkinson's disease, fibromyalgia, chronic pain, sleep disorders, autism or attention deficit hyperactivity disorder in a patient in need thereof that comprises administering to the patient a therapeutically effective amount of an NR2B receptor antagonist or a pharmaceutically acceptable salt thereof.

7. The method of claim 1 wherein the patient is a human.

8. The method of claim 1 wherein the NR2B receptor antagonist is a non-peptidal NR2B receptor antagonist.

9. The method of claim 1 wherein the NR2B receptor antagonist is an orally active NR2B receptor antagonist

10. The method of claim 1 wherein the NR2B receptor antagonist is a selective antagonist of the NR2B receptor.
